(19) United States
(12) Patent Application Publication
(10) Pub. No.: US 2006/0004266 A1
(43) Pub. Date: Jan. 5, 2006

(54) Bio-information processing apparatus and video/sound reproduction apparatus

(76) Inventors: Katsuya Shirai, Kanagawa (JP); Yoichiro Sako, Tokyo (JP); Toshiro Terauchi, Tokyo (JP); Makoto Inoue, Tokyo (JP); Masamichi Asukai, Kanagawa (JP); Yasushi Miyajima, Kanagawa (JP); Kenichi Makino, Kanagawa (JP); Motoyuki Takai, Tokyo (JP)

Correspondence Address: JAY H. MAIOLI;Cooper & Dunham LLP, 1185 Avenue of the Americas, New York, NY, 10036, US

(21) Appl. No.: 11/158,729

(22) Filed: Jun. 22, 2005

(30) Foreign Application Priority Data
Jul. 5, 2004 (JP) P2004-197791

(51) Int. Cl. A61B 5/00; A61B 5/02; A61B 5/08; A61B 5/04

(52) U.S. Cl. 600/300; 600/546; 600/500; 600/529

(57) ABSTRACT

A bio-information processing apparatus includes a single bio-information sensor for outputting a biological signal including a plurality of measured bio-information values of a subject, an analyzing circuit for analyzing the biological signal separating the bio-information values, and a circuit for estimating the psychological state and intensity of the psychological state of the subject from measured bio-information values included in a plurality of biological signals and from one of initial bio-information values and reference bio-information values.

### CROSS REFERENCES TO RELATED APPLICATIONS

The present invention contains subject matter related to Japanese Patent Application JP 2004-197797 filed in the Japanese Patent Office on Jul. 5, 2004, the entire contents of which are incorporated herein by reference.

### BACKGROUND OF THE INVENTION

1. Field of the Invention

The present invention relates to a bio-information processing apparatus and a video/sound reproduction apparatus.

2. Description of the Related Art

Recently, ideas have been proposed to detect a person's bio-information by using a bio-information sensor attached to the person's clothing and accessories, such as glasses, earrings, a necklace, a watch, or a jacket and to infer the person's psychology from the detected bio-information.

For example, there is a method of inferring a person's psychology from a fluctuation of the person's pulse rate (or heart beat rate). In this method, the subject wears an electrocardiograph or a pulse sensor to measure his or her pulse rate. By observing the fluctuation in the subject's pulse rate, the subject's tension or emotional change can be detected (for example, refer to Japanese Unexamined Patent Application Publication Nos. 7-323162 and 2002-23918).

Instead, heat rate or pulse rate can be measured by a sensor directly attached on the subject's finger or wrist or a sensor attached to a necklace, grasses, business cards, or a pedometer to infer a change in the subject's tension and/or emotion based on the measurements. There is also a method of estimating the synchronization between two people (degree of entrainment between two people) by measuring how well the pulse rates of the two people match when they are communicating (refer to Japanese Unexamined Patent Application Publication Nos. 11-4892 and 2002-112969).

There is also a method of inferring a person's psychology from a plurality of biological signals of, for example, optical blood flow, electrocardiographic activity, electrodermal activity, and skin temperature. When employing such a method, the subject wears a watch-type sensor to optically measure blood flow, electrocardiographic activity, electrodermal activity, and skin temperature. Then, from the measurements, a characteristic vector extracting the characteristics of each index is generated. The characteristic vector is compared with a plurality of emotional state values stored in a database in advance. In this way, the subject's psychology can be categorized into different psychological states, such as joy, relief, satisfaction, calmness, overconfidence, grief, dissatisfaction, anger, astonishment, fear, depression, and stress (for example, refer to Japanese Unexamined Patent Application Publication No. 2002-112969).

If the subject's psychological state can be inferred from such measurements, for example, if an operator of a device suffers a disability that makes it difficult for him or her to operate the device, an operation environment most desirable for the operator's psychological state can be provided automatically.

### SUMMARY OF THE INVENTION

However, it is often difficult to infer one's psychology by employing the above-described methods. For example, there are facial expressions, such as ‘astonishment’ and ‘confusion,’ that are difficult to distinguish from each other. Furthermore, it is known that one's pulse rate shows the same kind of change when the level of arousal is high while the level of valence is either positively high (i.e., when the subject is feeling pleasure) or negatively high (i.e., when the subject is feeling displeasure). For this reason, valence inferred from pulse rate when arousal is high may be incorrect.

By combining a plurality of bio-information items, the accuracy of the estimation can be increased. However, to obtain a plurality of bio-information values, a plurality of sensors is required and the apparatus for obtaining a plurality of bio-information values becomes large and complex. Furthermore, the psychological burden on the object becomes great.

The main object of the above-described methods is to merely categorize one's psychology from bio-information. Therefore, the intensity of one's psychological state, such as “extreme pleasure” or “moderate pleasure,” cannot be measured correctly.

The apparatuses and method according to embodiments of the present invention can infer a subject's psychological state and the intensity of the psychological state from an output signal from a single bio-information sensor. Moreover, according to the psychological state of the subject, the apparatuses provide an environment, including images and sounds, optimal to the subject's psychology.

A bio-information processing apparatus according to an embodiment of the present invention includes a single bio-information sensor for outputting a biological signal including a plurality of measured bio-information values of a subject, an analyzing circuit for analyzing the biological signal, separating the measured bio-information values from the biological signal, and outputting the measured bio-information values, and an estimating circuit for estimating the psychological state and intensity of the psychological state of the subject from the measured bio-information values and from one of initial bio-information values and reference bio-information values.

The bio-information processing apparatus according to an embodiment of the present invention is capable of inferring a subject's psychological state and the intensity of the psychological state from a plurality of bio-information values to obtain the values of arousal and valence. Then, images and sound can be reproduced in accordance with the obtained results such that the user's psychological state is maintained at an optimal state. Since a plurality of bio-information values are obtained from an output from a single bio-information sensor, the subject's burden can be reduced and the apparatus can be simplified.

### BRIEF DESCRIPTION OF THE DRAWINGS

FIG. 1 is a schematic diagram of a video/sound reproduction apparatus according to an embodiment of the present invention;

FIG. 2 illustrates a method of processing an output from a sensor according to an embodiment of the present invention;

FIG. 3 is a flow chart showing a control flow according to an embodiment of the present invention; and

FIG. 4 illustrates another graph representing an embodiment of the present invention.

### DESCRIPTION OF THE PREFERRED EMBODIMENTS

[1] Video/sound Reproduction Apparatus

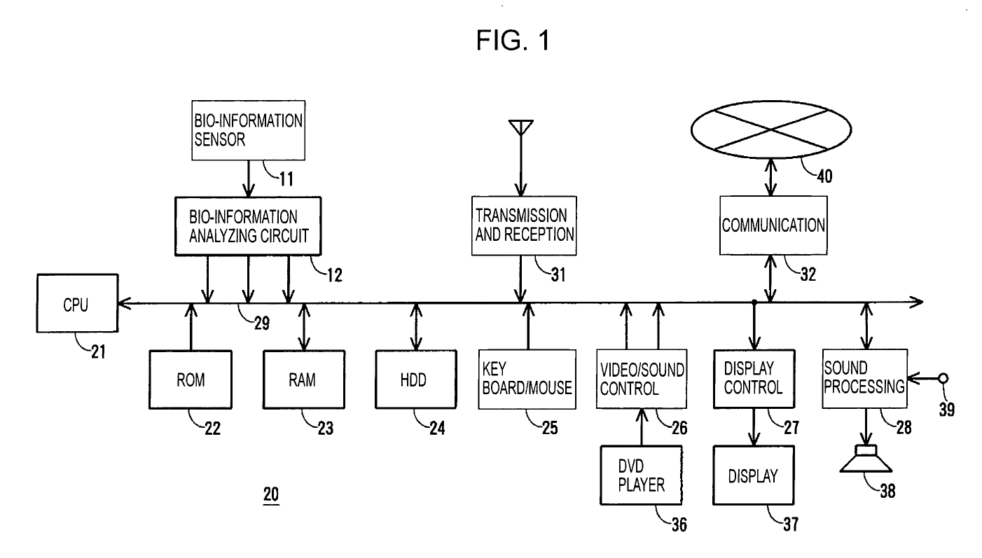

FIG. 1 illustrates a video/sound reproduction apparatus according to an embodiment of the present invention. The video/sound reproduction apparatus obtains different types of bio-information values of a user (subject) by a single bio-information sensor, determines arousal and valence, which are indices representing the user's psychological state from the obtained bio-information values, and changes the reproduced images and sound in accordance with the arousal and valence.

Accordingly, the video/sound reproduction apparatus includes a bio-information sensor 11 for obtaining a plurality of bio-information values of a user. The bio-information sensor 11 may be a noncontact-type sensor for obtaining bio-information of the user without making physical contact with the user or may be a wearable noncontact-type sensor for obtaining bio-information of the user by making physical contact with the user.

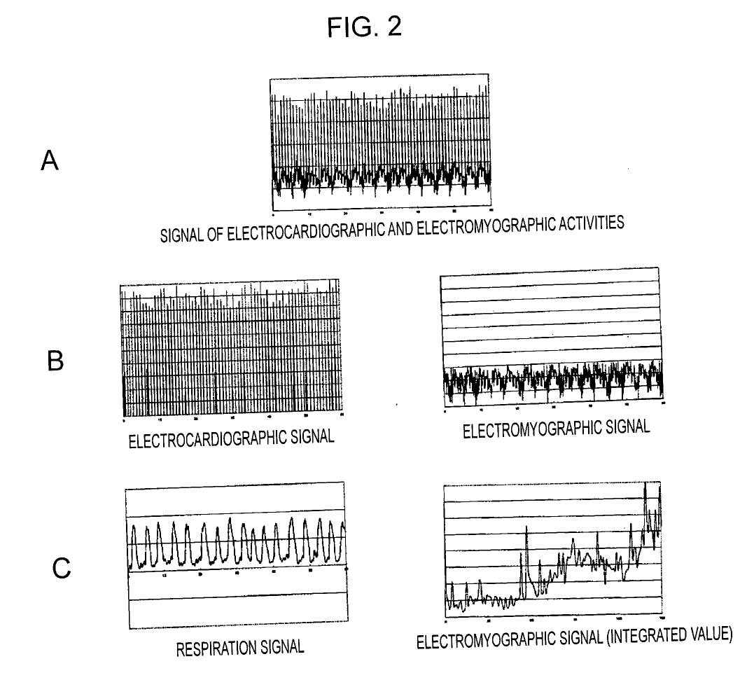

When the bio-information sensor 11 is a noncontact-type sensor, the sensor may be constituted by a sheet-type piezoelectric device and a sheet-type strain gauge or a card including a piezoelectric device and a strain gauge. Then, the bio-information sensor 11 is disposed in, for example, a pocket on the user's left chest. In this way, the bio-information sensor 11, for example, can output a signal simultaneously including an electromyographic (EMG) signal and an electrocardiographic signal, as illustrated in FIG. 2A.

When the bio-information sensor 11 is a contact-type sensor, for example, an electrocardiograph and an electromyograph may be attached to the user's chest to output a signal simultaneously including an electromyographic signal and an electrocardiographic signal.

The output from the bio-information sensor 11 is supplied to a bio-information analysis circuit 12. In this case, the electrocardiographic signal and the electromyographic signal included in the output of the bio-information sensor 11 are distributed in a frequency band below 2 Hz and around 40 Hz, respectively. In the bio-information analysis circuit 12, the output from the bio-information sensor 11 is filtered and the output is separated in frequency bands including the electrocardiographic signal and the electromyographic signal, as illustrated in FIG. 2B. The separated electrocardiographic signal and electromyographic signal are supplied to a microcomputer 20.

Since the user's cardiac pulsation fluctuates due to the user's respiration, the intervals between the R-wave of the electrocardiographic signal also fluctuate. In other words, the fluctuation of respiration (i.e., respiratory sinus arrhythmia (RSA)) is superimposed on the electrocardiographic signal. Therefore, by analyzing the electrocardiographic signal, a signal representing the user's respiration can be obtained indirectly.

In the bio-information analysis circuit 12, the fluctuation of the separated R-wave in the electrocardiographic signal over time is determined and the power spectrum is obtained by FFT (fast Fourier transform) processing. The peak in the frequency band between 0.15 to 0.40 Hz of the power spectrum represents the respiration component. By repeating the above-described processing by carrying out FFT processing in 5-second intervals, the fluctuation of the respiration component over time can be determined, and, in other words, a respiration signal can be obtained indirectly. The respiration signal is also supplied to the microcomputer 20.

In the microcomputer 20, and the electrocardiographic signal, the arousal and valence of the user are computed from the electromyographic signal, and the respiration signal supplied to the microcomputer 20. In accordance with the computed results, desirable video image and sound are reproduced.

More specifically, the microcomputer 20 includes a central processing unit (CPU) 21, a read only memory (ROM) 22 storing various programs, and a random access memory (RAM) 23 used as a work area, wherein each of the units are mutually connected via a system bus 29.

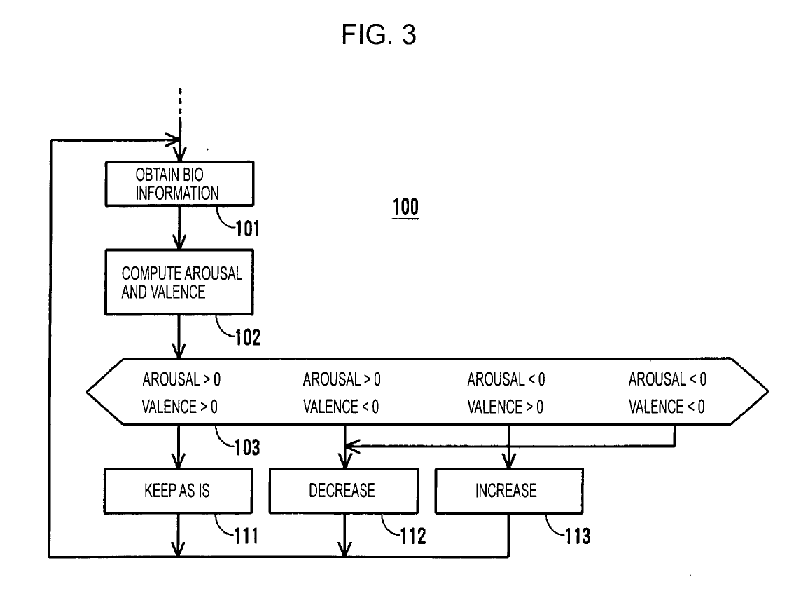

In this case, the ROM 22 stores, for example, a routine 100, as illustrated in FIG. 3, as part of a program executed by the CPU 21. Details of the routine 100 will be described below. The routine 100 is configured to control an image signal or a sound signal in accordance with the user's bio-information such that video image and sound can be perceived by the user with pleasure. As illustrated in FIG. 3, the routine 100 according to an embodiment is part of a program, and this part includes only the processes that are included in the scope of the present invention.

The microcomputer 20 includes a hard disk drive 24 used as a mass storage device and a user interface 25, such as a keyboard or a mouse. Both the hard disk drive 24 and the user interface 25 are also connected to the system bus 29. According to this embodiment, a digital versatile disk (DVD) player 36 is provided as a source of image signals and sound signals. The DVD player 36 is connected to the system bus 29 via a video/sound control circuit 26.

In this case, the video/sound control circuit 26 is capable of controlling the image signal reproduced by the DVD player 36 to modify the conditions, such as contrast, brightness, hue, and saturation of color of a displayed image and controlling the reproduction speed of the DVD player 36. Furthermore, the video/sound control circuit 26 controls the sound signal reproduced by the DVD player 36 to control the volume, frequency characteristics, and reverberation of the reproduced sound.

The system bus 29 is connected to a display 37 via a display control circuit 27. An image signal output from the video/sound control circuit 26 is converted into a display signal by the display control circuit 27. This display signal is supplied to the display 37. A sound processing circuit 28 is connected to the system bus 29 to supply a sound signal to a speaker 38 via the sound processing circuit 28 and to supply a sound signal from a microphone 39 to the microcomputer 20 via the sound processing circuit 28.

Bio-information and other data of the user collected by the video/sound reproduction apparatus and other apparatuses may be transmitted between each apparatus by connecting the system bus 29 to a transmission and reception circuit 31 and a communication circuit 32. The communication circuit 32 is connected to other networks, such as the Internet 40.

According to the above-described structure, an image signal and a sound signal are reproduced by the DVD player 36 by operating the user interface 25. The image signal is supplied to the display 37 via the video/sound control circuit 26 and the display control circuit 27 so as to display an image on the display 37. Similarly, the sound signal is supplied to the speaker 38 via the video/sound control circuit 26 and the sound processing circuit 28 to play sound from the speaker 38.

At this time, the CPU 21 executes the routine 100 to compute the user's arousal and valence in response to the image displayed on the display 37 and the sound played from the speaker 38. Based on the computed values, the image and sound are controlled so that they are perceived by the user with pleasure.

More specifically, when the routine 100 is executed, first in Step 101, bio-information collected by the bio-information sensor 11 is sent to the microcomputer 20 via the bio-information analysis circuit 12. Then, in Step 102, arousal and valence are computed based on the bio-information sent to the bio-information analysis circuit 16 in Step 101. The computation method will be described below. Both arousal and valence are obtained by computation in analog values that may be either positive or negative values.

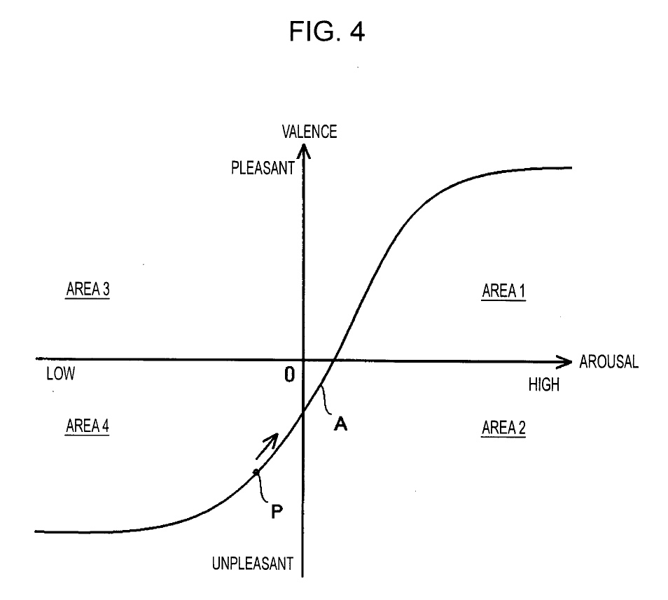

Subsequently, the process proceeds to Step 103. In Step 103, the signs (positive or negative) of the value of arousal and valence obtained in Step 102 are determined. Then, the next step in the process is determined in accordance with the combination of the signs of the values. In other words, since both arousal and valence may be either a positive value or a negative value, when arousal and valence are plotted on two-dimensional coordinate axes, the graph illustrated in FIG. 4 is obtained. According to this graph:
in Area 1, arousal>0 and valence>0 (arousal is high and the user is in a state of pleasure);
in Area 2, arousal>0 and valence<0 (arousal is high and the user is in a state of displeasure);
in Area 3, arousal<0 and valence>0 (arousal is low and the user is in a state of pleasure); and
in Area 4, arousal<0 and valence<0 (arousal is low and the user is in state of displeasure).

When the values of arousal and valence fall into Area 1, it is assumed that the user is perceiving the image and sound pleasantly, and the process proceeds from Step 103 to Step 111. In Step 111, the image signal and the sound signal supplied to the display 37 and the speaker 38, respectively, are not modified, and then the process proceeds to Step 101. In other words, when the values of arousal and valence fall into Area 1, it is inferred that the user is satisfied with the image and sound and thus the reproduction conditions of the image and sound are not changed.

However, when the values of arousal and valence fall into Area 2, it is assumed that the user is perceiving the image and sound with displeasure, and the process proceeds from Step 103 to Step 112. In Step 112, to remove the user's displeasure, for example, the level of the direct current and/or alternate current of the image signal sent to the display 37 is lowered to lower the brightness and/or contrast of the image displayed on the display 37. Similarly, for example, the level of the sound signal sent to the speaker 38 is lowered and/or the frequency characteristics of the sound signal are modified to lower the volume of the sound output from the speaker 38, weaken the low and high frequency bands of the sound signal, and/or weaken the rhythm of the sound. Then, the process proceeds to Step 101.

If the condition set in Step 112 continues for a predetermined period of time, this means the values of arousal and valence are not being improved and the user is still experiencing displeasure. In such a case, for example, the reproduction of image and sound can be terminated in Step 112.

When the values of arousal and valence fall into Area 3, the process proceeds from Step 103 to Step 113. In Step 113, contrary to Step 112, the user's degree of pleasure can be increased and/or feelings can be elevated, for example, by increasing the level of the direct current and/or alternating current of the image signal sent to the display 37 to increase the brightness and/or contrast of the image displayed on the display 37. Similarly, for example, the level of the sound signal sent to the speaker 38 can be increased and/or the frequency characteristics of the sound signal can be modified to increase the volume of the sound output from the speaker 38, strengthen the low and high frequency bands of the sound signal, and/or emphasize the rhythm of the sound. Then, the process proceeds to Step 101.

For example, if the user sets the video/sound reproduction apparatus to ‘sleeping mode’ using the user interface 25, images and sound can be reproduced so that the values of arousal and valence stay in Area 3 since images and sounds in this area will not interfere with the user's sleep.

When the values of arousal and valence fall into Area 4, it is assumed that the user is perceiving the image and sound with displeasure, and the process proceeds from Step 103 to Step 112. The user's displeasure is removed in the same manner as in the case in which the value of arousal and valence fall into Area 2.

Accordingly, by executing the routine 100, image and sound can be reproduced in a manner such that the user will always perceives the image and sound with pleasure.

In this way, the above-described video/sound reproduction apparatus is capable of inferring a user's psychological state and the intensity of the psychological state by using a plurality of bio-information values collected by the bio-information sensors 11 to obtain the values of arousal and valence of the user. Then, images and sound can be reproduced in accordance with the obtained results such that the user's psychological state is maintained at an optimal state. Since a plurality of bio-information values are obtained by the output from a single bio-information sensor, the user's burden can be reduced and the apparatus can be simplified.

[2] Computing Arousal and Valence

In which area in the graph, illustrated in FIG. 4, the values of arousal and valence of the user falls can be determined by the processes described below in sections [2-1] and [2-2]. If, for example, the present values of arousal and valence of the user are at a point P, in FIG. 4, it can be determined in which direction along the curved line A including the point P the values of arousal and valence will change based on previous change history of the values.

Accordingly, the best image and sound for the user's psychological state can always be provided. Moreover, if the user is in a positive psychological state, this positive state can be maintained and if the user is in a negative psychological state, this state can be improved.

[2-1] Computing Arousal

Arousal can be determined from the electrocardiographic signal and the respiration signal and can be determined from the deviation of the measured respiratory rate and pulse rate of the user from initial or standard values. The bio-information sensor 11 used to measure the user's respiratory rate and pulse rate may be either noncontact-type sensors or contact-type sensors. Arousal can be computed using the formulas below:
Arousal=Rᵣₘ−Rᵣᵣ (1)
where, Rᵣₘ represents the measured respiration rate per unit time and Rᵣᵣ represent the initial or standard respiration rate per unit time, or
Arousal=Pᵣₘ−Pᵣᵣ (2)
where, Pᵣₘ represents the measured pulse rate per unit time and Pᵣᵣ represent the initial or standard pulse rate per unit time. Formula (2) may be used to compute arousal even when the heart rate is being used as pulse rate.
[2-2] Computing Valence

Valence can be computed, for example, from an electromyographic signal by applying the following Formula (3):
Valence=∫|V_{emg}(t)|dt−V_{emg}_{_{—}}ᵢₙᵢₜ (3)
where V_{emg} represents the magnitude of the fluctuation of the measured value of electromyographic activity and V_{emg}_{_{—}}ᵢₙᵢₜ represents the integrated value (initial value) of the magnitude of fluctuation of electromyographic activity, or
Valence=∫|V_{emg}(t)|dt−V_{emg}_{_{—}}_{ref} (4)
where V_{emg}_{_{—}}_{ref} represents the magnitude of the fluctuation of the integrated value (reference value) of electromyographic activity.

The positive value of valence is determined based on the electromyographic measurements taken from the cheek bone muscle and the negative value of valence is determined based on the electromyographic measurements taken from the corrugator muscle or the orbicularis muscle.

[3] Other Descriptions

A pressure sensor may be used as the bio-information sensor 11. In such a case, a pressure sensor containing a pneumatic sensor in an air-tight soft bag, as described in Japanese Unexamined Patent Application Publication No. 2001-145605, may be used. The above-described bio-information sensor 11 was disposed in the chest area of the user. However, the bio-information sensor 11 may be disposed anywhere on the user so long as a signal simultaneously including an electromyographic signal, and an electrocardiographic signal or a pulse signal is obtained.

Moreover, when changing an image signal and/or a sound signal based on the user's psychological state and when its intensity is being inferred from the measurements, as described above, the reproduction speed, volume, color, and/or content of images and/or sound may be modified. The image signals and sound signals modified based on the measured bio-information may be recorded.

As a recording medium, the hard disk drive 24, an optical disk, a magneto-optical disk, a magnetic tape, a hard disk, a semiconductor memory, or an integrated chip (IC) card may be used. The optical disk may be a compact disk (CD), a CD-Recordable (CD-R), a CD-ReWritable (CD-RW), a mini disc, a DVD-Recordable (DVD+R), a DVD-ReWritable (DVD+RW), a DVD random access memory (DVD-RAM), or a Blu-ray Disc. As described above, image signals and sound signals can be modified based on bio-information. A setting may be provided for selecting whether or not to accept the modification.

As described above, the image and/or sound reproduction conditions are controlled based on computed values of arousal and valence. Instead of controlling images and/or sound reproduction based on the values of arousal and valence, the environment of the user, such as the user's house, office, and relationship with other people, can be assessed, or usability of products can be assessed. Furthermore, the results of computing arousal and valence can be displayed as graphs and numerals.

It should be understood by those skilled in the art that various modifications, combinations, sub-combinations and alterations may occur depending on design requirements and other factors insofar as they are within the scope of the appended claims or the equivalents thereof.

1. A bio-information processing apparatus, comprising:
a single bio-information sensor for outputting a biological signal including a plurality of measured bio-information values of a subject;
an analyzing circuit for analyzing the biological signal, separating the plurality of measured bio-information values from the biological signal, and outputting the plurality of measured bio-information values; and
an estimating circuit for estimating a psychological state and intensity of the psychological state of the subject from the plurality of measured bio-information values and from one of initial bio-information values and reference bio-information values.

2. The bio-information processing apparatus according to claim 1, wherein the bio-information sensor is in contact with the subject.

3. The bio-information processing apparatus according to claim 1, wherein at least one of the plurality of measured bio-information values is one of respiration rate, pulse rate, and electromyographic activity of the subject.

4. The bio-information processing apparatus according to claim 1, wherein the psychological state of the subject is at least one of emotion, mood, arousal, and valence.

5. The bio-information processing apparatus according to claim 4, wherein the arousal is estimated from a change in at least one of a pulse rate and a respiration rate of the subject.

6. The bio-information processing apparatus according to claim 4, wherein the valence is estimated from a change in electromyographic activity of the subject.

7. A video/sound reproduction apparatus, comprising:
reproduction means for reproducing at least one of an image signal and a sound signal;
a single bio-information sensor for outputting a biological signal including a plurality of measured bio-information values of a subject;
an analyzing circuit for analyzing the biological signal, separating the plurality of measured bio-information values from the biological signal, and outputting the plurality of measured bio-information values; and
an estimating circuit for estimating a psychological state and an intensity of the psychological state of the subject from the plurality of measured bio-information values and from one of initial bio-information values and reference bio-information values.
modification means for modifying at least one of the image signal and the sound signal reproduced by the reproduction means in accordance with results estimated by the estimating circuit.

8. The video/sound reproduction apparatus according to claim 7, wherein the bio-information sensor is in contact with the subject.

9. The video/sound reproduction apparatus according to claim 7, wherein at least one of the plurality of measured bio-information values is one of respiration rate, pulse rate, and electromyographic activity of the subject.

10. The video/sound reproduction apparatus according to claim 7, wherein the psychological state of the subject is at least one of emotion, mood, arousal, and valence.

11. The video/sound reproduction apparatus according to claim 10, wherein the arousal is estimated from a change in at least one of a pulse rate and a respiration rate of the subject.

12. The video/sound reproduction apparatus according to claim 10, wherein the valence is estimated from a change in electromyographic activity of the subject.

13. The video/sound reproduction apparatus according to claim 7, wherein the modification means modifies at least one of reproduction speed, volume, color, and content of at least one of the image signal and the sound signal.

14. The video/sound reproduction apparatus according to claim 7, further comprising:
recording means for recording at least one of the plurality of measured bio-information values, and a sound signal and an image signal modified based on the plurality of measured bio-information values.

15. The video/sound reproduction apparatus according to claim 14, wherein the recording means is one of an optical disk, a magneto-optical disk, a magnetic tape, a hard disk, a semiconductor memory, and an integrated circuit card.

16. The video/sound reproduction apparatus according to claim 15, wherein the optical disk is one of a compact disk, a compact disk-Recordable, a compact disk-ReWritable, a mini disc, a digital versatile disk-Recordable, a digital versatile disk-ReWritable, a digital versatile disk random access memory, and a Blu-ray Disc.

17. The video/sound reproduction apparatus according to one of claims 7 to 16, wherein a user is capable of selecting whether to approve or forbid the modification of at least one of an image signal and a sound signal based on the plurality of bio-information values.

18. A video/sound reproduction apparatus, comprising:
a reproduction unit for reproducing at least one of an image signal and a sound signal;
a single bio-information sensor for outputting a biological signal including a plurality of measured bio-information values of a subject;
an analyzing circuit for analyzing the biological signal, separating the plurality of measured bio-information values from the biological signal, and outputting the plurality of measured bio-information values; and
an estimating circuit for estimating a psychological state and intensity of the psychological state of the subject from the plurality of measured bio-information values and from one of initial bio-information values and reference bio-information values.
a modification unit for modifying at least one of the image signal and the sound signal reproduced by the reproduction means in accordance with the results estimated by the estimating circuit.
